(19) United States
(12) Patent Application Publication
(10) Pub. No.: US 2013/0310627 A1
(43) Pub. Date: Nov. 21, 2013

(54) SYSTEM FOR PROVIDING MAGNETIC FIELD

(71) Applicant: PROJECT 18 LLC, New York, NY (US)

(72) Inventors: David Franco, Brooklyn, NY (US); Daniel Laniado, New York, NY (US)

(73) Assignee: Project 18 LLC, New York, NY (US)

(21) Appl. No.: 13/711,824

(22) Filed: Dec. 12, 2012

Related U.S. Application Data
(63) Continuation-in-part of application No. 13/476,076, filed on May 21, 2012.

(51) Int. Cl. A61N 2/00; A61N 2/06; H01F 7/02

(52) U.S. Cl. CPC A61N 2/004; H01F 7/02; A61N 2/06
USPC 600/15; 335/219

(57) ABSTRACT

A system for providing a magnetic field wherein the system has
a magnet exhibiting a magnetic surface field of 600 to 5000 Gauss; said magnet incorporated into a securing article; and
said securing article is constructed and arranged to attach to a wearer.

### INDEX TO RELATED APPLICATIONS

This application is continuation-in-part of U.S. patent application Ser. No. 13/476,076 filed May 21, 2012, the disclosure of which is incorporated herein by reference in its entirety.

### BACKGROUND OF THE INVENTION

### Field of the Invention

Many alternative medicine and holistic medicine practitioners use magnet therapy or magnetic therapy. The theory behind magnetic therapy is that the magnetic fields increase blood flow to a particularized area based on the known phenomenon that red blood cells contain iron, a magnetic element. Although there are disputes over the efficacy of magnetic therapy, and in the United States the Food and Drug Administration will not allow specific claims to magnetic therapy to be made in products, there still are many products introduced that purport to provide magnetic therapy. One of the difficulties in providing magnetic therapy is that there are a seemingly unlimited amount of magnetic materials that exist in our world. If one would to truly attempt to analyze the effect of magnetic therapy on a patient it would require a seemingly exhaustive search of magnetic materials and studies of their properties and results. The present invention has discovered a particularized magnetic characteristic that provides the desired magnetic therapy effect. The present invention does not purport to circumvent any particular FDA regulation but merely to say that amongst practitioners that utilize magnetic therapy and assert its efficacy, the magnetic properties described herein appear to be advantageous.

### SUMMARY OF THE INVENTION

While there is no shortage of commercially available articles relating to magnetic therapy, it remains difficult to select a desirable article due to lack of reliable specifications.

The present invention addresses the vast uncertainty by providing a particularized magnet and magnetic field observed to be advantageous over other known and used magnets.

In one embodiment, the invention is a system for providing a magnetic field said system comprising:

a magnet exhibiting a magnetic surface field of 1,550 Gauss;
said magnet incorporated into a securing article; said securing article constructed and arranged to attach to a wearer.

The article is constructed and arranged to connect about the circumference of an arm, a leg, or a skull as a compression armband, leg band, or headband. The compression force imparted directional from above the skin to the direction of the skin surface. In a preferred embodiment, the magnet has a Brmax of about 10,800 Gauss and the BHmax is about 28 MgOe.

In one embodiment, more than one magnet is used and the relative placement of successive magnets imparts a continual magnetic field along a substantially horizontal plane above or along the skin surface.

The magnetic field of the invention is imparted by a magnetic solid, semi solid, or fluid.

Preferably, the magnetic field is imparted by a NdFeB magnet coated with a surface coating of Ni—Cu—Ni.

In a preferred embodiment, substantially all of an outer surface of said NdFeB magnet is coated with said surface coating of Ni—Cu—Ni.

Also contemplated in the present invention is a method of imparting a magnetic field above the skin surface, said method consisting of:

providing a NdFeB magnet coated with a surface coating of Ni—Cu—Ni, said magnet having a magnetic surface field of 1,550 Gauss, a Brmax of about 10,800 Gauss and a BHmax of about 28 MgOe;
securing said magnet to a support article, wherein in said support article is selected from the group consisting of, a headband, an armband, a wristband, a leg band, or an ankle band and imparts a compression force from above skin surface towards skin surface;
attaching said support article to the portion of the body in which support article is configured for attachment, said attaching exposes adjacent skin surface to a particularized magnetic field.

Additionally, it has been discovered that the pole orientation can enhance efficacy in the current invention.

The North Pointing Pole (Positive)

In one embodiment, magnetic system of the present invention utilizes small North Pointing magnets. Pole Disc Magnets of 600 to 5000 gauss, which possesses strengthening and stimulating qualities, is used to provide increased energy. The magnet does not have to touch the skin, but can hover over the contour of the body, with the North Pointing Pole facing the body. This is the suggested magnetic technology used for vigorous activities.

The South-Pointing Pole Magnet (SPPM) withdraws energy from the source, which is used on an in_amed or tight muscle to give relief. Alternatively, an overly relaxed muscle needs more energy and consequently the North Pointing Pole Magnet (NPPM).

Magnetic technology increases blood flow and circulation.

In one embodiment, magnets targeting the centripetal zone are fused or otherwise incorporated into clothing products according to the present invention. This incorporation optimizes magnetic delivery, does not significantly interfere with motion and allows an athlete to perform at their optimum performance level.

The system and method of the present invention includes placement of magnetic technology in the apparel in the centripetal zone which receives magnetic waves and further provides for application of magnetic waves when energy is exerted by an athlete, thus producing waves of force through the centi poles for targeted point-of-contact cell rejuvenation.

The centripetal zone takes advantage of centripetal force, which is continual, as long as the athlete continues to move with magnetic waves being emitted.

In one embodiment, the invention is a system for providing a magnetic field said system comprising:

a pole disc magnet having a north and south magnetic field exhibiting a magnetic surface field of 600 to 5000 gauss;
said magnet incorporated into a securing article, whereby said incorporation includes the north pole surface of said magnet facing a user's body;
said securing article constructed and arranged to attach to a wearer.

Additionally, the invention includes, a method of imparting a magnetic field above the skin surface, said method consisting of:

selecting a muscles muscle group on a human body in which application of a magnetic filed is desired;
selecting a magnet having a magnetic field corresponding to said selected muscle or muscle group;
providing a pole disc magnet having a magnetic north and south magnetic field, said magnet having a magnetic surface field of 600 to 5000 Gauss, a Brmax of about 10,800 Gauss and a BHmax of about 28 MgOe;
securing said magnet to a support article, wherein in said support article positions said magnet with said north magnetic field facing a user's body;
attaching said support article to the portion of the body in which support article is configured for attachment, said attaching exposes adjacent skin surface to a particularized magnetic field.

### BRIEF DESCRIPTION OF THE SEVERAL VIEWS OF THE DRAWING

FIG. 1A is a front view of the present invention.

FIG. 1B is a cross section along section lines B-B of FIG. 1A.

FIG. 2 is a side view of one embodiment of the present invention.

FIG. 3 is a partial section view from section lines A-A of FIG. 2.

FIG. 4 is a perspective view of one embodiment of the present invention.

FIG. 5 is a side view of one embodiment of the present invention.

FIG. 6 is a side view of one embodiment of the present invention.

FIG. 7A is a front view of one embodiment of the present invention.

FIG. 7B is a partial cross section along section line C-C from FIG. 7A.

FIG. 8 is a front view showing various positions for magnet placement according to the present invention.

FIG. 9 is a rear view of a person showing magnet placement according to the present invention.

FIG. 10 is the front view of a shirt showing various magnet placements according to the present invention.

FIG. 11 is a rear view of a shirt showing various magnet placements according to the present invention.

FIG. 12 is a front view of another embodiment of a shirt showing magnet placement according to the present invention.

FIG. 13 is a rear view of an embodiment of shirt showing magnet placement according to the present invention.

FIG. 14 is the rear view of a pair of pants showing magnet placement according to the present invention.

FIG. 15 is a side view showing magnet placement according to the present invention.

FIG. 16 is the front view of a pair of pants showing magnet placement according to the present invention.

FIG. 17 is a partial left arm sleeve view of a shirt showing magnet placement according to the present invention.

FIG. 18 is a partial upper arm sleeve portion of a shirt showing magnet placement according to the present invention.

FIG. 19 is a partial lower leg portion from pants showing magnet placement according to the present invention.

FIG. 20 is a partial wrist sleeve view showing magnet placement according to the present invention.

FIG. 21 is a front view of a pair of shorts showing magnet placement according to the present invention.

FIG. 22 is a rear view of a pair of shorts showing magnet placement according to the present invention.

FIG. 23 is an enlarged view showing magnet placement within garment material according to the present invention.

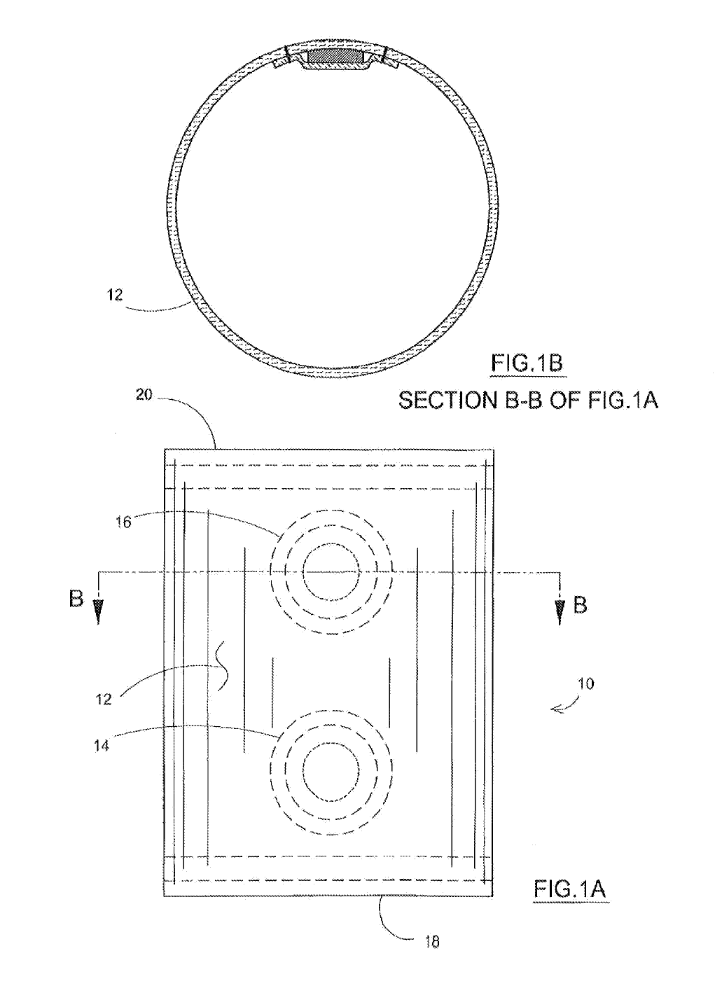

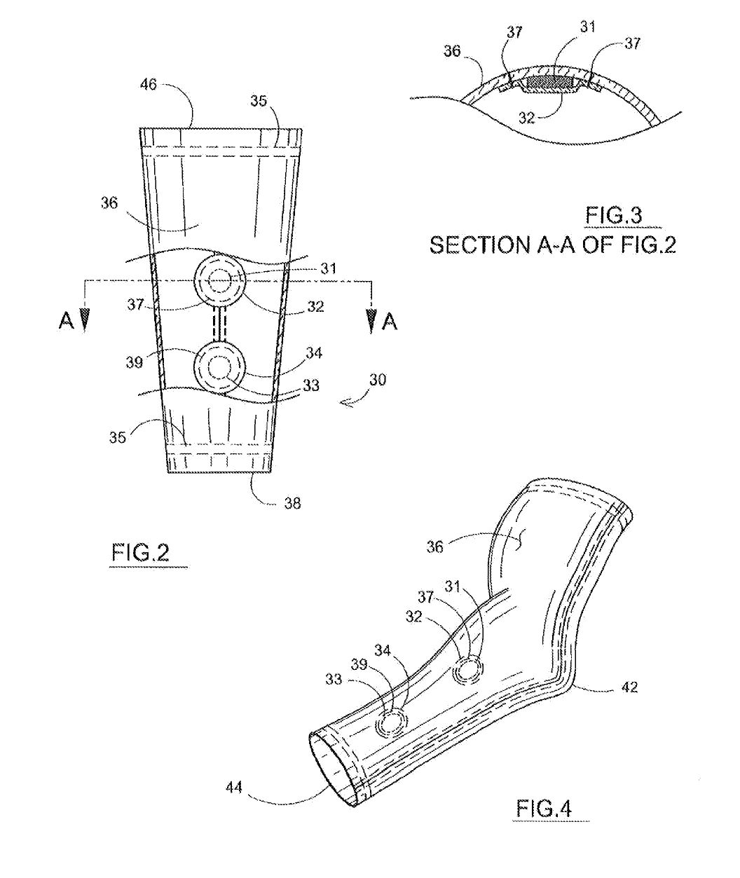

### DETAILED DESCRIPTION OF THE PREFERRED EMBODIMENT

The article of the present invention is constructed and arranged to provide magnetic therapy to the skin surface of a patient. Magnets are sometimes used in therapy with the intention of increasing blood flow. As is known, red blood cells contain iron, a magnetic metallic element. The article of the present invention secures a magnet in place above the surface of the skin of a patient in order to impart a localized magnetic field.

In one embodiment of the present invention a support structure 12 has magnet 17 secured thereto. Magnet 17 is held in place by magnet support 16. In some embodiments magnet support 16 is further secured to article 12 through a securing means 18. Securing means 18 can be any acceptable means such as stitching, adhesives and the like. Furthermore, support 16 can be any acceptable material that will secure magnet 17 into a fixed position. Materials can include various types of fabrics, tapes and the like. In one embodiment, the article 12 can have a first magnet 17 and a second magnet 21. When second magnet 21 is utilized a second magnet support 19 secures magnet into position and support 19 is attached to article 12 through attachment support 14. Article 12 will have a first edge 20 and a second edge 18 whereby an interior cavity is defined such that article 12 is worn about either arms or legs of a user.

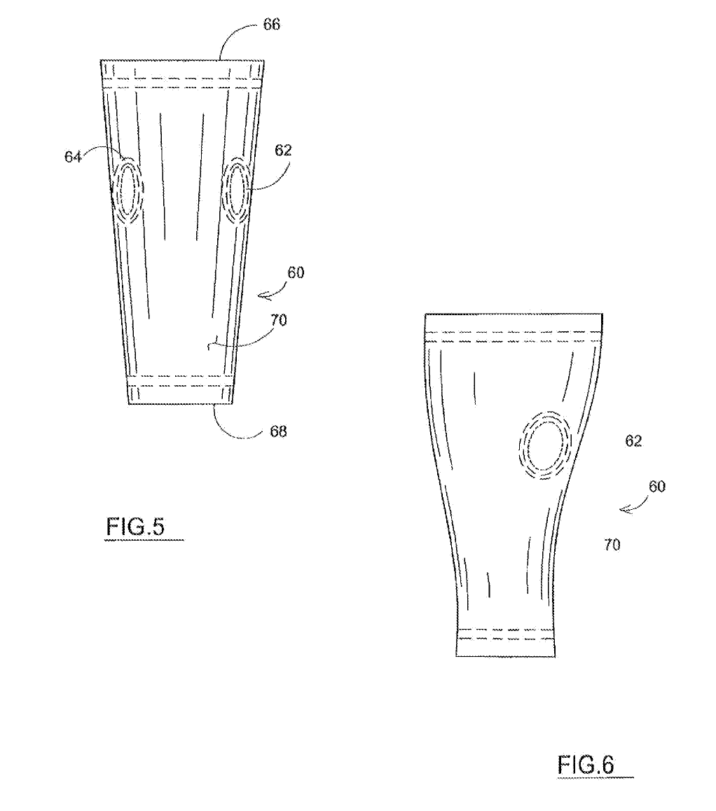

An embodiment such as those shown in FIGS. 5 and 6 is constructed and arranged to be placed over a user's leg. Magnet 63 is held in place by support 61 and ultimately attached to article 70 through attaching support 62. In this embodiment, there is a first edge 66 and a second edge 68 which defines an inner region which is formed such that article 70 is wrapped around a user's leg. A user will insert their foot into the inner region and slide article 70 until article 70 is in a desired position around a user's leg.

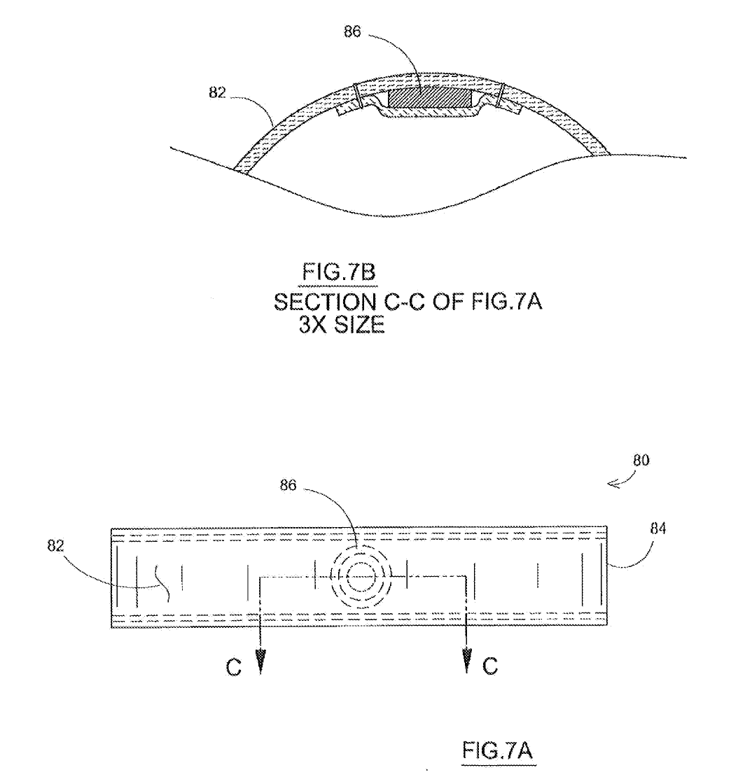

As seen in FIG. 7A, the article of the present invention can be constructed and arranged in the form of a headband to secure a magnets in position about the skull. Magnet 85 is secured on article 80 by magnet support 86 and secured to article 80 surface 82 through support connector 87. Surface 82 is constructed and arranged to have a particular length such that it will secure about the circumference of the user's head, generally around the forehead. IN a preferred embodiment, magnet 85 is positioned in any location about the head. One or more magnets can be incorporated such that magnets are placed on any of forehead, temples, rear of skull, and combinations thereof. The article of the present invention can be constructed in any configuration as described herein with the impetus being the placement of a magnet or magnets with particular properties on or in close proximity to, the skin surface in a desired location.

As generally understood, magnetic therapy has been applied in many different manners. However, the present invention has discovered not only a particularized article for providing magnetic therapy but has identified out of the vast variable magnetic materials a particular type of magnet suited for the therapy design. A neodymium magnet (also known as NdFeB, NIB, or Neo magnet), the most widely-used type of rare-earth magnet, is a permanent magnet made from an alloy of neodymium, iron, and boron to form the Nd₂Fe₁₄B tetragonal crystalline structure. It has been discovered that a magnet comprising NdFeB, grade N28 which is coated with Ni—Cu—Ni has been found to be particularly useful. This magnet has a pull force in case one of 1.67 pounds and a pull force in case two of 7.12 pounds. It exhibits a surface field of 1550 Gauss. The Residual Flux Density (Brmax) of the material, is essentially the magnetic field density inside the magnet material. The quality of magnetic materials is best stated by the Maximum Energy Product (BHmax), measured in MegaGauss Oersted (MGOe).

In a preferred embodiment, the desired magnetic field is Brmax of about 10,800 Gauss and the BHmax is about 28 MgOe. It has been discovered that magnets with these specifications have a particularly enhanced effect when being used in physiological magnetic therapy. It is understood that the magnetic therapy has an underlying goal to increase blood flow, and more particularly concentrate red blood cells in the area within close proximity of the magnets. The particular magnets described herein have been determined after extensive experimentation to have magnetic field properties particularly conducive for physically magnetic therapy.

As generally understood by the figures presented herein, it is desired that a magnet be positioned such that it remains substantially stationary on a user. The present invention is not limited to any particular embodiment such as the armband, head band, or leg band, demonstrated in the figures. Further, the invention is not limited to the placement of a single or double magnet as demonstrated in the figures. The figures are illustrative of several possible embodiments but are not meant to be exhaustive. Based on the magnetic properties it is contemplated that therapy should be applied for a time period between approximately one and ten hours. In a preferred embodiment therapy is applied for a time period of about one to eight hours. In an additional embodiment, flexible magnetic material may be use such that a magnet can be elongated and curve around joints such as fingers, elbows, shoulders, knees and the like. When flexible magnets are used they are still contemplated to exhibit the same magnetic properties as described herein. In still a further embodiment, a fluid or semisolid magnetic material can be incorporated into articles of the present invention. In these types of embodiments as in embodiments incorporating flexible magnets the magnets can be formed to provide therapy about flexible areas such as fingers, elbows, shoulders, knees and the like. What remains important is if fluid type material is incorporated into an article of the present invention, the alternate magnetic characteristics are contemplated to be the same as described herein. When referring to the magnetic characteristics particular attention is paid to the pull force and surface field BRmax and BHmax characteristics that are incorporated by the magnets.

To the extent that magnetic therapy is disputed, the present invention relates to the introduction of a magnetic field to the skin surface of a patient. The magnetic field will have the characteristics as disclosed herein and the location will be determined by the article holding a magnet or magnetic material in position in said desired location.

The illustrated embodiments, as shown in the Figs., are of elastic and or compression articles constructed and arranged to support one or more magnets. It is additionally contemplated that compression force, as is commonly known and understood in the filed of elastic and or/compression articles such as arm bandages, leg bandages and headbands (shown in the Figs.), apply pressure along the surface of the skin in areas under and in close proximity to the bandage. The present invention further contemplates, in the system of the present invention, configuring the article to be an elastic or compression bandage.

It is further contemplated that by securing a magnet above the surface of the skin, combined with a compression article effectuating said securing, resultant magnetic fields will pass through the skin with minimal disruption. This placement and securing with a compression force thus ensures magnetic field is delivered in a desired are while also maintaining the characteristics of the magnetic field imparted.

The present invention further is directed to a system and method whereby a muscle or muscles group is targeted and magnet selection is dependent thereon. In this embodiment, a magnet having opposing north south poles is used and the magnetic north is pointed towards the body.

In an alternative embodiment, as desired, the south pole surface is pointed towards the body (e.g. proximal in direction).

By using particularized polarity in the application of magnetic fields, the present invention contemplates increased efficacy in the administration of magnetic fields to particularized muscle groups.

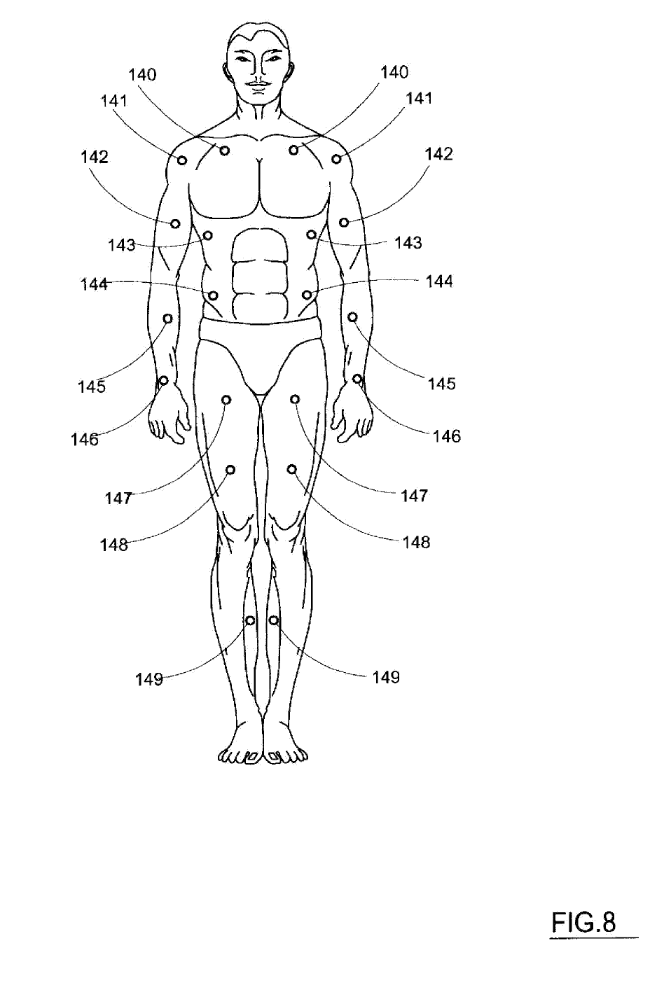

In embodiments as shown in FIGS. 8-22, a person 100 has various known muscle regions whereby according to the system and method of the present invention it is desirable to impart magnetic fields.

As best seen in FIG. 8 these regions include selective placement of magnets in any one or combination of pectoral muscles 140, front deltoids muscles 141, bicep muscles 142, external abdomen oblique 143, lower abdomen oblique 144, inner forearm 145, wrist flexor 146, upper quadriceps 147, lower quadriceps 148, inner calves 149.

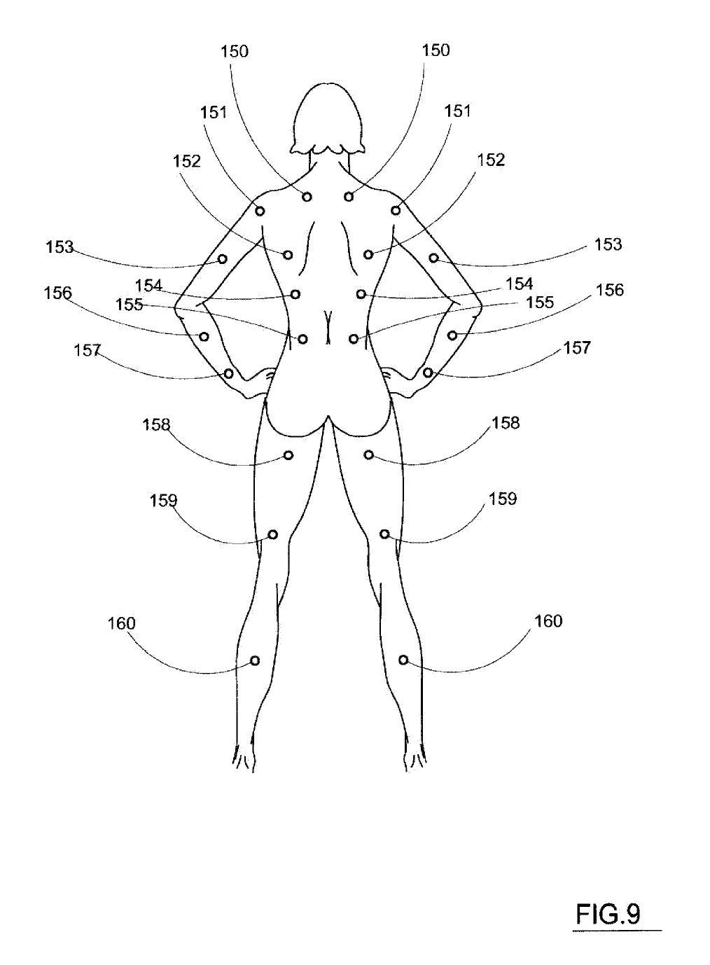

Further as seen in FIG. 9 muscle groups targeted include trapezoid 150, deltoids 151, teres major 152, triceps 153, middle lateral muscles 154, lower lateral muscles 155, outer forearm 156, rear flexor 157, upper hamstring 158, lower hamstring 159 and calves 160. The system and method includes contemplating providing magnets in at least one of these particular muscle regions.

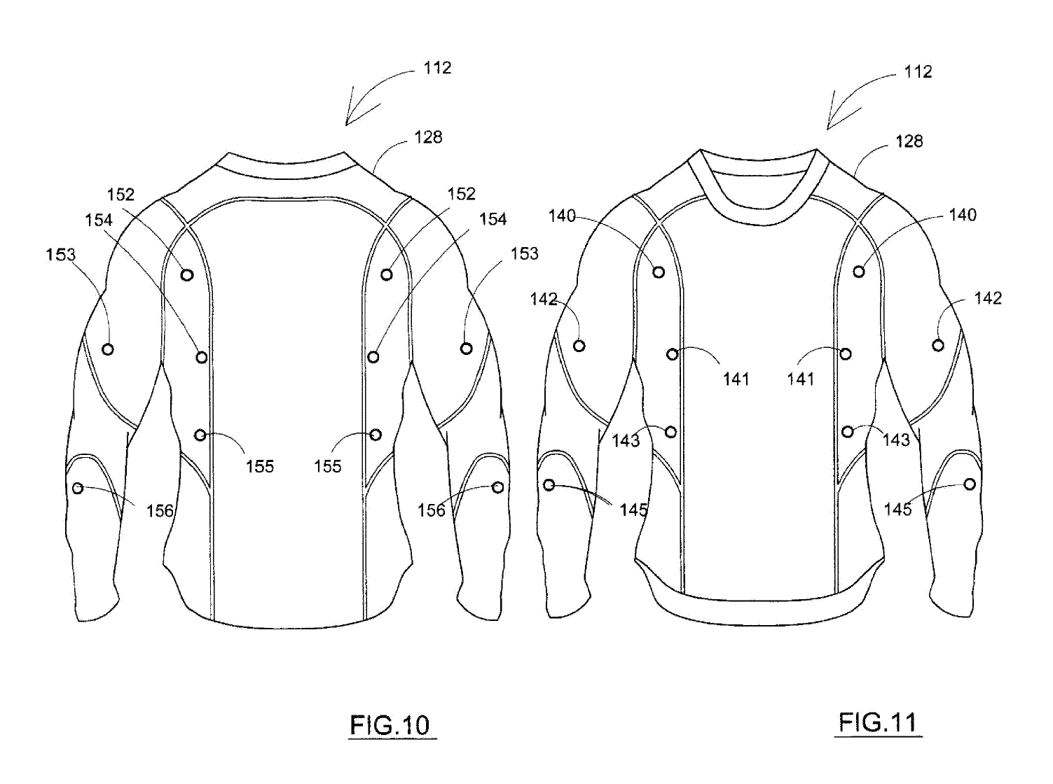

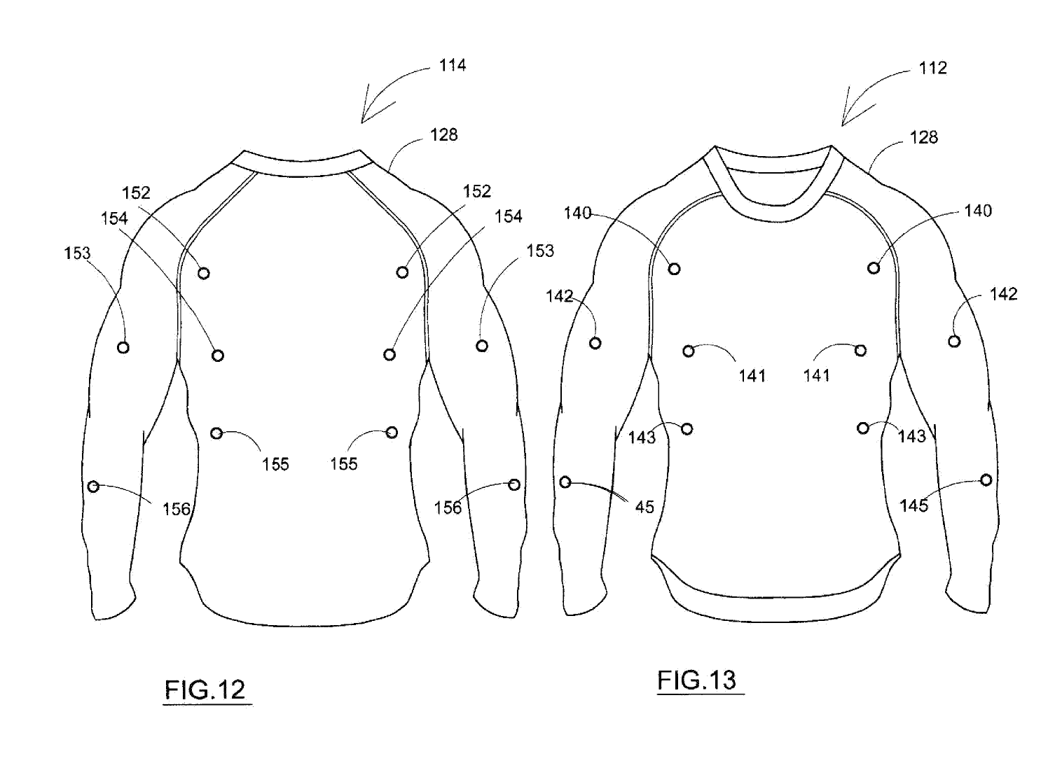

Further, the system and method of the present invention includes specific and particularized combinations of muscle regions selected in which magnetic fields are to be applied. In one embodiment as shown in FIGS. 10 and 11 a first shirt configuration 12 has selective placement of magnets as designated by muscle group placement relative to the shirt when a user wears the shirt. Placement includes any one or combination of teres major 152, triceps 153, mid lateral muscles 154, lower lateral muscles 155 or forearm muscles 156. Additionally placement includes any one or more of pectoral muscles 140, deltoids muscles 141, bicep muscles 142, external abdominal obliques 143 and inner forearm 145. FIGS. 12 and 13 show a second shirt configuration 14 with magnet placement along muscle groups as indicated.

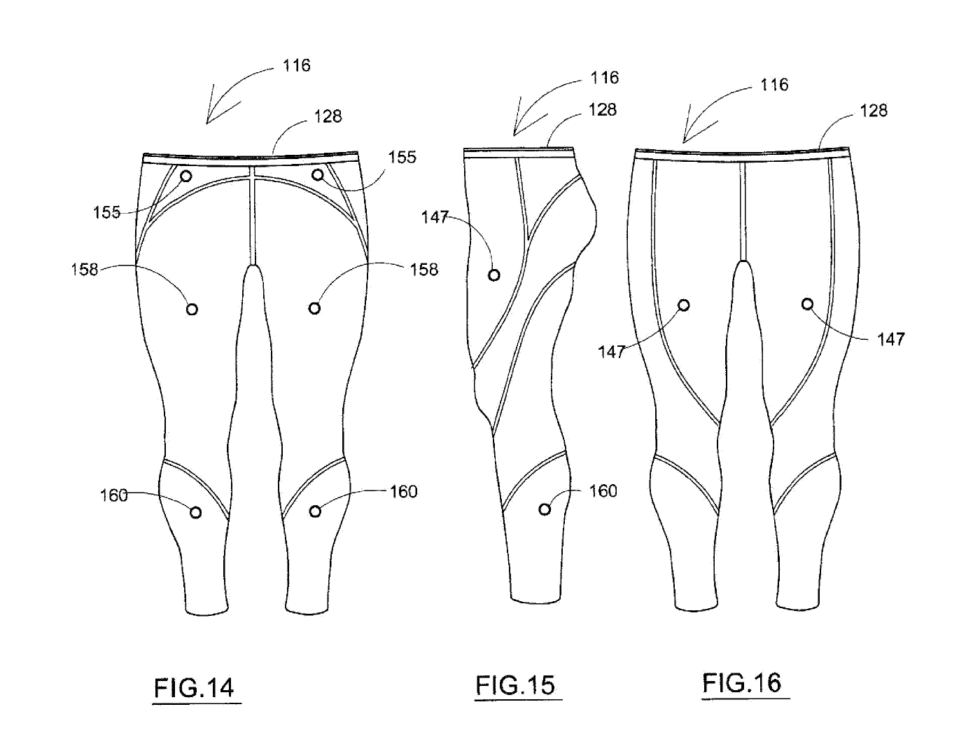

FIGS. 14 through 16 show a pair of pants with magnets placed above muscles groups as indicated mid-lower lateral muscles 155, upper hamstring muscle 158, calf muscles 160 upper quadriceps 147.

In one embodiment, when magnets are incorporated into the fabric of a garment, such as shirt, pants, shorts, and the like, it is contemplated that the garment is form fitting and exerts pressure in the proximal direction towards the wearer.

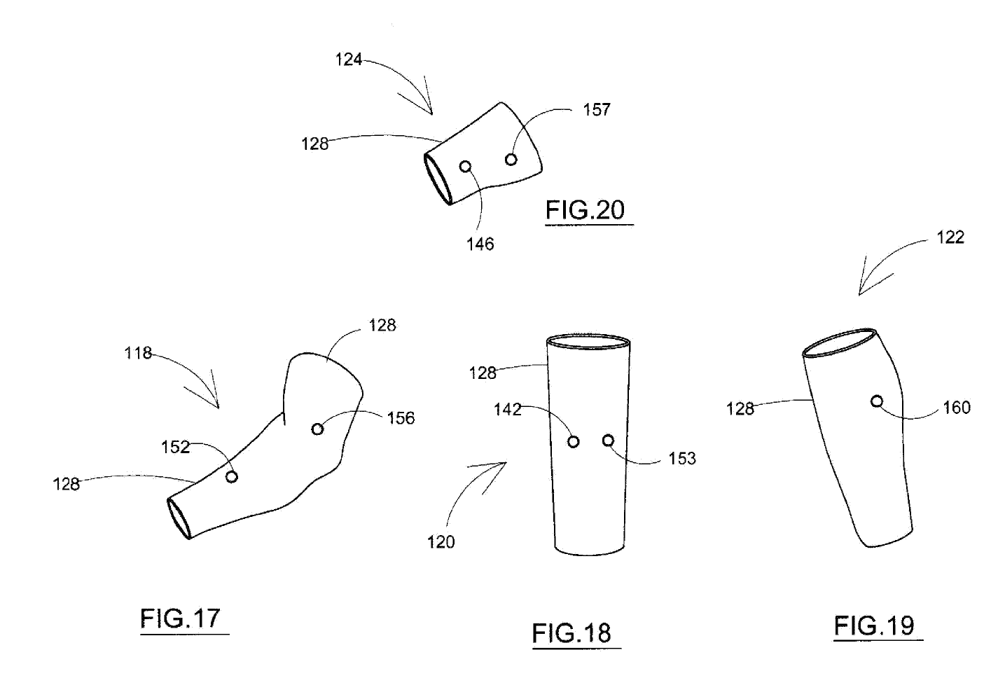

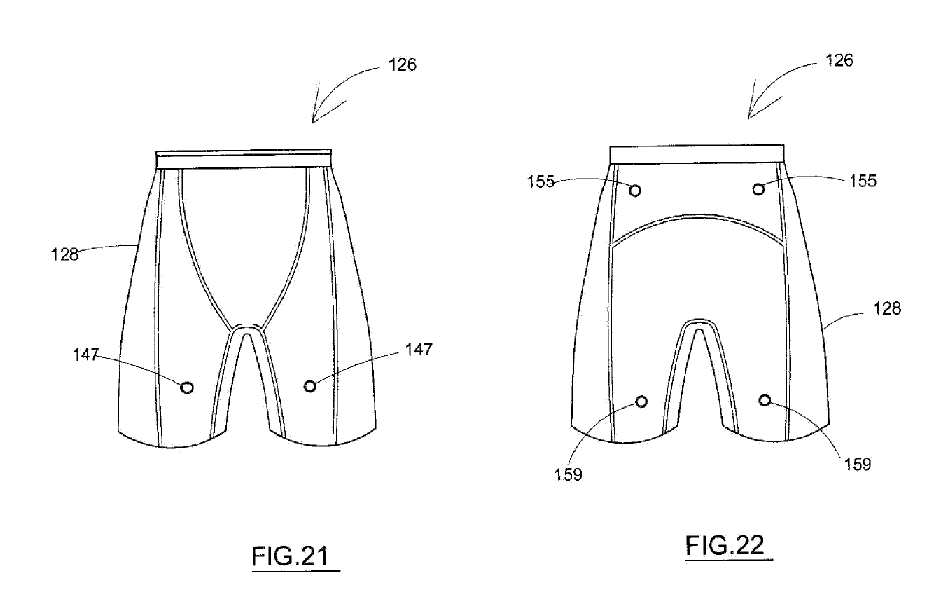

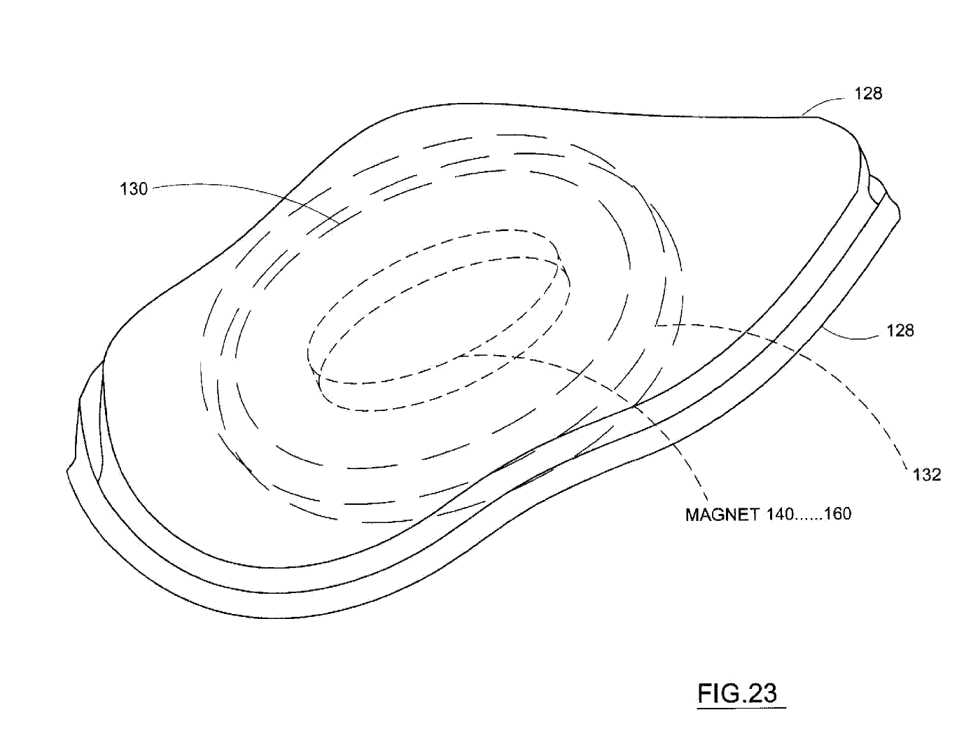

FIGS. 17-20 show partial views whereby specific muscles groups are targeted in the lower arm as shown in FIG. 17, the upper arm as shown in FIG. 18, the lower leg as shown in FIG. 19 and the wrist area as shown in FIG. 20. In one embodiment as shown in FIGS. 21 and 22 a pair of shorts 26 has magnet placement relative to muscle groups being upper quadriceps 147, lower lateral muscles 155 and upper hamstrings 158. As shown in FIG. 23 and generally understood, magnets 40 according to the present invention are embedded within fabric 28 in a magnet pocket 121 such that their position is relatively fixed. In one embodiment, magnet is secured pocket fastener 130 to ensure magnets do not move in an undesirable manner. This facilitates desired placement for imparting magnetic fields at particular muscle locations.

In one embodiment each individual magnet has a non-magnetic overlay 132 on the portion of the magnet facing outward (distal) from the wearer. This means essentially the magnetic field is only imparted inward (proximal) towards the wearer and no magnetic field is imparted outward from the wearer. The overlay is any appropriate material that blocks or inhibits magnetic field from being imparted outward (distal) from the wearer. Essentially, magnets with the overlay are effectively monopole or single pole magnets.

The present invention contemplates the non-magnetic portion configured to limit undesirable attraction between individualized garments while they are hanging on a rack or folded and stacked during non-use.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

1. A system for providing a magnetic field said system comprising:
a pole disc magnet having a north and south magnetic field exhibiting a magnetic surface field of 600 to 5000 gauss, wherein said magnet has a Brmax of about 10,800 Gauss and the BHmax is about 28 MgOe;
said magnet incorporated into a securing article, whereby said incorporation includes the north pole surface of said magnet facing a user's body;
said securing article constructed and arranged to attach to a wearer; and
a non-magnetic overlay in a distal direction from a wearer, formed of a material that blocks or inhibits magnetic field from being imparted outward (distal) from the wearer, said overlay imparting magnetism as a monopole.

2. The system of claim 1 wherein said article is constructed and arranged to connect about the circumference of an arm, a leg, or a skull.

3. (canceled)

4. The system of claim 1 wherein more than one magnet is used.

5. The system of claim 1 wherein more than one magnet is used and the relative placement of successive magnets impart a continual magnetic field.

6. The system of claim 1 wherein said magnetic field is imparted by a magnetic solid, semi solid, or fluid.

7. The system of claim 1 wherein said magnetic field is imparted by a NdFeB magnet coated with a surface coating of Ni—Cu—Ni.

8. The system of claim 7 wherein substantially all of an outer surface of said NdFeB is coated with said surface coating of Ni—Cu—Ni.

9. The system of claim 1 wherein said article imparts a compression force from above skin surface towards skin surface.

10. A method of imparting a magnetic field above the skin surface, said method consisting of:
selecting a muscles muscle group on a human body in which application of a magnetic filed is desired;
selecting a magnet having a magnetic field corresponding to said selected muscle or muscle group;
providing a pole disc magnet having a magnetic north and south magnetic field, said magnet having a magnetic surface field of 600 to 5000 Gauss, a Brmax of about 10,800 Gauss and a BHmax of about 28 MgOe;
securing said magnet to a support article, wherein in said support article positions said magnet with said north magnetic field facing a user's body;
attaching said support article to the portion of the body in which support article is configured for attachment, said attaching exposes adjacent skin surface to a particularized magnetic field; and
providing a non-magnetic overlay in a distal direction from a wearer, said overlay formed of a material that blocks or inhibits magnetic field from being imparted outward (distal) from the wearer, said overlay imparting magnetism as a monopole.
